(19) United States
(12) Patent Application Publication
(10) Pub. No.: US 2004/0166556 A1
(43) Pub. Date: Aug. 26, 2004

(54) Analytic device for the detection and quantification of fermentative cultured activities

(76) Inventor: Dennis Roy Cullimore, Regina (CA)

Correspondence Address: Walter A. Rodgers, 880 North Island Drive, Atlanta, GA, 30327, US

(21) Appl. No.: 10/369,865

(22) Filed: Feb. 21, 2003

(51) Int. Cl. C12Q 1/04; C12M 1/34

(52) U.S. Cl. 435/34; 435/287.1

(57) ABSTRACT

A test device provides an enclosed gas entrapment system for the safe determination of both the presence and rate of occurrence of fermentation gases where the targeted microorganisms were both present and active. The gas entrapment device has a density greater than the liquid culture medium within the chamber and therefore settles to the base of the chamber and includes slots in the vertical walls of the device which allow the passage of fermentative microorganisms into and out of the device during the period of culture and examination. When there has been the generation of gases entrapped within the device, the density of the device becomes compromised forcing the device to elevate in the liquid fermenting medium to form an indication that gases have been produced in the sample by the microorganisms and that organisms are present.

### FIELD OF THE INVENTION

This invention relates generally to a method and apparatus for performing microbiological analysis, and is more particularly concerned with an appropriately sequenced cultural method and apparatus for the screening of a sample to determine the quantifiable presence of fermentative activity generating gases in the presence of the organism of choice.

### DISCUSSION OF THE PRIOR ART

Recent advances in microbiology have engendered many techniques useful to the engineer, microbiologist and various health specialists. A large number of these tests are conducted daily both in the field and in the laboratory. With the increasing awareness of the greater diversity in sources of microbially driven compromises of systems and causes of infections, there is an accelerating demand for the effective screening of substances, notably water and pathologic material to ensure accurate determination of cause for a given economically significant compromise or clinically important infection. However, due to the time required for these tests, and the cost of such tests, complete testing is not economically feasible so that the tests may only be run on substances under suspicion because of inferential evidence.

Those skilled in the art will realize that an incipient problem may not manifest itself in the natural state for one or more of a variety of reasons; the contamination may become obvious after some inferences in the natural system. By way of example, bacteria may be present in small quantities, but may not be readily detectable through some understood biochemical or cultural function through the inadequate ability of the conventional cultural devices to display some form or other of affirmative occurrence in a manner that appropriately allows a quantitative determination of the event.

The prior art includes cultural systems primarily utilizing liquid media within which a fermentative function is detected by the production of gaseous products. These gaseous products of fermentation were determined to be significant to the determination of the nature of the dissimilatory processes possessed by some of the important groups of microorganisms that has since become embodied into the standard cultural methodologies for the determination of the nature of fermentative microorganisms. Today the diagnosis of the enteric bacteria in samples is achieved with the aid of determinative procedures for the detection of gaseous end products of fermentation processes within specific forms of culturing devices. A variety of entrapment devices were developed to detect these gases and it was the Durham's tube that has become the most widely accepted. The concept was generated in the early years of the twentieth century and involved the simple placement of an inverted glass test tube in the liquid cultural medium that also filled this inverted tube. The presence of gas could be visually determined by a growing pocket of gas entrapped in the top region of the tube. The prior art leans on the simple nature of the Durham's tube to detect fermentation gases and act as the primary diagnostic system for the qualitative detection of fermenting microorganisms of concern. This art has been developed in particular to detect the coliform bacteria in samples since these bacteria are considered to be an acceptable indicator of health risks associated with the sample under test.

Traditionally the method for the qualitative confirmation of selected fermentative microorganisms was by the application of various organic substrates with the confirmation being achieved by the appropriate demonstration of gas production in specific media. Classical science commonly directed the use of an inverted glass tube in the medium to entrap some of the gaseous products within a gas filled pocket that could be viewed as confirmatory of the fermentative activities. This device became known as the Durham's tube and has been adapted to quantitative studies through the determination of which of various dilutions generated observable gas production (therein considered positive indicators). The distribution of these said indicators could now be statistically interpreted to project a most probable population estimate for the number of targeted fermentative microorganisms within the original sample.

Attempts have been made to improve the Durham's tube including one method that involved replacing the glass used in the traditional tube with a semi-permeable material that allowed the freer movement of microbes and chemicals between the inverted tube and the surrounding medium. This modification had the advantages of the gas entrapment causing a decline in density of the device leading to an elevation resulting from increased buoyancy generated by these gases. The time to such an elevation was further found to link in a direct manner to the size of the active population involved in generating said gases. This invention represents a new method of control for the operation of the U.S. Pat. No. 5,187,072 described above but involves a unique and separate method of control.

Information disclosing prior art can be found in the following articles:

PASTEUR, L (1857) Mémoire sur la fermentation appelée lactique. Comptes rendus de l'Académie des sciences, Volume 45, 913-916.

KLUYVER, A. J (1924) Eenheid en verscheidenheid in do strofwisseling der microbien'. Chemisch Weekblad, Volume 21, 266

CULLIMORE, D. R (2000) Practical Atlas for Bacterial Identification, Lewis Publishers, Boca Raton Fla. pp 209.

TORTORA, G. J., FUNKE, B. R. and C. L. CASE (2001) Microbiology, an introduction, 7^{th}. Edition, Benjamin Cummings, Addison Wesley Longman Inc. New York.

### SUMMARY OF THE INVENTION

The present invention provides a method whereby the detection of the fermentation gases resulting from microbial activities within a culture vessel can be detected with greater qualitative convenience than the present Durham's tube method and also, where the time lag to the observation of confirmed gas entrapment, to determine quantitatively the size of the active targeted microbial population within the sample. Determination of the time lag may be based on either the presence of the gases within the present device changing the characteristics of the device in a definable manner or causing the relocation of the device as the density of the device declines with the admission of gases to the internal body of the device. Detection may be visual, spectrophotometric or involve some form of electromagnetic anomaly to the field in, and around, the device.

The present device provides the form of an inverted tube similar in form to the Durham's tube to allow the entrapment of the fermentation gases. This device differs from the said tube in that it is constructed of a density adjusted material that causes the device to sink in liquid culture media when the device is evacuated of air. A second difference with the said tube is that there are vertical openings (slots) in the lower parts of the wall of the inverted device that allows the free access and release of microorganism and chemicals between the inside void of the device and the surrounding volume of culture medium containing the sample under examination. These slots act in the manner of allowing the two environments within, and without, the device to more freely exchange and equilibrate to the betterment of the fermentative processes occurring within the culture vessel containing the present device.

The device described and claimed as a part of this invention above functions as a result of a set of circumstances that result in the elevation of the device as a result of the increasing buoyancy of the device due to the gases accumulating within the head space of the device. Elevation would, however, remain dependent upon the density of the device and the density of the surrounding medium and would occur at that point when the accumulating gases reduce the density of the device to less than that of the surrounding medium. Precision therefore becomes in part a product of the differences between the density of the device and density of the surrounding medium wherein the device should initially have a greater density than the medium at the start of the test. A critical set of interactive factors are the density of the liquid medium and the thimble, the population of microorganisms that are active and able to ferment the selective medium at the incubation temperature with the production of accumulative gases, and the relationship of the total volume (TV) of the medium to the volume of the medium entrapped within the voids (VV) in device confined at the top and by the sides. It may be surmised, by those familiar with the art that the amount of gas that may be entrapped by the device will increase where the VV approaches that of the TV. Such an event could cause a faster elevation of the device due to the greater entrapment of gases. This in turn has the potential to affect the time lag length prior to elevation and influence the precision with which the time lag could be quantitatively converted to a total enteric population (TEP). Two key elements to improve the precision would be to set a suitable range for the TV:VV ratio that would result in a common ability to entrap the fermented gases, and set a suitable tolerance that the density of the device will be greater than the liquid medium within which the device is sunk. For the purposes of an example the TV:VV ratio should be set at between 1:0.2±0.05 and the density of the device should be within the range of 0.05±0.02 g/mL above the ambient density of the liquid medium at room temperature.

Given that sample can have differing densities then the device has the option to be constructed of greater or less dense materials to meet the needs for testing the specific sample such as fresh-water and sea-water and allow a similar time lag to the elevation of the present invention having compensated for density. To maintain position within the culture vessel, the present invention may have a series of extensions that may take the form of vertical vanes to assure that the device moves in a specific manner within the culture vessel. To allow spectrophotometric or electromagnetic monitoring of the position of the present invention as it moves through buoyancy created by the entrapment of gases within said device, the material for the construction of the device may be modified to significantly detect changes in the characteristics of light or electromagnetic fields that would be associated with the movement of the device resulting from the entrapment of fermentation gases. It should also be noted that the form of the culture vessel within which the determination is being undertaken may be modified to improve the determination of this event. By the exploitation of the time lag information generated by the entrapment of gases it is possible through to comparative studies to determine the population of the microorganisms that are of interest and are able to generate fermentative gases under the cultural conditions applied.

### BRIEF DESCRIPTION OF THE SEVERAL VIEWS OF THE DRAWINGS

These and other features and advantages of the present invention will become apparent from consideration of the following specification when taken in conjunction with the accompanying drawings in which:

FIG. 1 is a vertical diametric cross-section of a culture chamber having a test device therein made in accordance with the present invention, and including the final volume of medium presented as a liquid broth.

FIG. 2 is a modified form of the device shown in FIG. 1 showing the effect of fermented gases have entered and caused, by way of an example, the vertical relocation of the present invention.

FIG. 3 is a vertical side-view of the device as shown in FIG. 1 but illustrating two forms in which positioning vanes can be placed on the present invention to allow more precise positioning within the culture chamber and to act as ballasting where greater density of the material composing the present invention are required to assure the detection of a determinable quantity of gas.

FIG. 4 is a modified version of FIG. 2 in which the typical light pathways are shown within an apparatus to allow the detection of the movement of the present invention as a result of shifts in buoyancy due to gas entrapment.

FIG. 5 is similar to FIG. 4 except that the detection of the movement of the present invention as a result of shifts in buoyancy due to gas entrapment is recorded by shifts in the electromagnetic fields induced in, and around, the present invention.

FIG. 6 is a composite of, and similar to FIG. 2 in which floatable device has been incorporated into the present invention positioned within the device to improve detection by any of the herein stated methodologies.

### DETAILED DESCRIPTION OF THE INVENTION

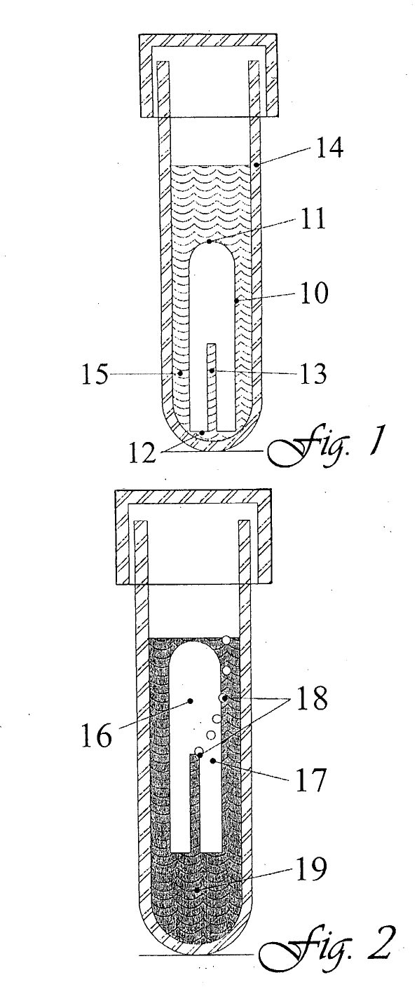

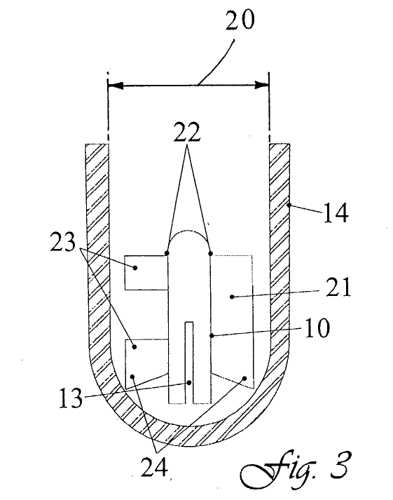

Referring now more particularly to the drawings, and to those embodiments of the invention here presented by way of illustration, the embodiments of the invention shown in FIG. 1 includes the test device 10 within a culture chamber 14. The physical arrangement is such that the test device 10 is set vertically filled with the culture medium 15 is sunk by virtue of the higher inherent density of the device 10 than the medium 15. A critical feature of the test device 10 includes the admission of one or more slots 13 in the lower part of the vertical walls of the device and a closed top 11 that retains any gases generated within the device during the test period. To assure that microorganisms from the culture medium 15 can enter the device to cause gas formation, the under side of the device 12 is open as well as the slots 13 on the lower wall of the device. The device 10 can be saturated with the medium 15 by either steam sterilizing the culture chamber 14 when capped in which case the air entrained within the device 10 is replaced by steam that condenses to fill device 10; or the culture chamber can be inverted which leads to the entrained air bubbling out of device 10 and being replaced with the culture medium 15. It should be noted that the slot 13 has a width that is large enough to allow microorganisms to enter and leave the test device 10 at the start, and during the early stages, of the test but does, however, become plugged with microbial biomass where an aggressive occlusive population of bacteria are present and active in the sample. Under such circumstances the whole length of the test device can become filled with fermentation gases adding to the buoyancy of the test device 10.

Where the culture medium 15 includes a sample containing microorganisms that can generate gases by fermentation then the manner in which these gases are detected is shown in FIG. 2. Here, the test device 10 becomes filled from the top down to create a gas-filled zone 16 where the culture medium is being displaced downwards (17) until the surplus gases begin to escape 18 through the slots 13. This fermentative activity is commonly accompanied with clouding as a result of the growth in the biomass and the culture medium now becomes clouded 19 and the gas, where seated in a device that is transparent, such as a glass Durham's tube, becomes difficult to determine visually.

In cases where the culture chamber 14 has a much larger diameter 20 than the test device 10 then there is a risk that the test device 10 may lean out of vertical sufficiently to become jammed. FIG. 3 illustrates a methodology to prevent this happening to the test device 10 through application of a single full length vane 21 connected to the body of the test device 10 by a suitable means 22 that will retain the position of the vane with respect to the said vane. Advantages in controlling the density or positioning of the test device 10 may be better achieved by the use of several partial length vanes 23. These vanes may be arranged vertically along the sides of the test device in at least three positions vertically to assure that the said test device is centrally positioned. In either event, these central extensions of the test device 10 in the form of vanes allow the said device to occupy a suitable position within the culture chamber 14 such as at the central axis. In order to reduce the risk of the test device 10 becoming biologically attached to the floor of the culture chamber 14 the invention may also include extensions downwards 24 on the lower side of the vanes that are likely to come into contact with the floor of the said culture chamber.

The nature of the clouding event 19 as a result of biological activity renders it difficult on some occasions to observe the presence of gases in the traditional sunken glass Durham's tube and even of the test device 10 itself unless it is constructed with either a distinct and contrasting color to the that of the clouded medium 19. As an example black may provide a suitable distinguishing color for visual determination of the test device 10 under such circumstances.

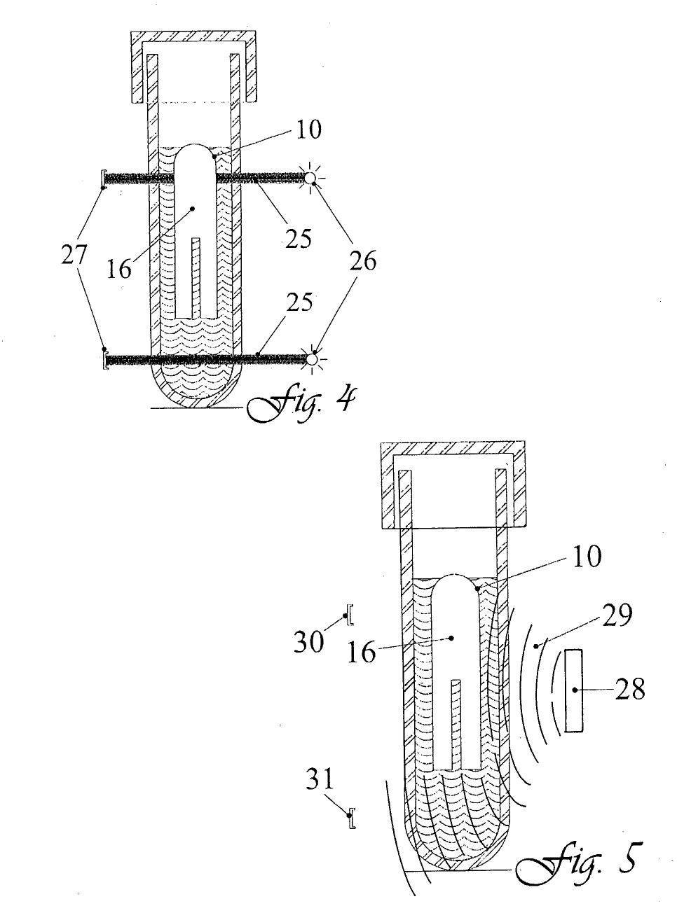

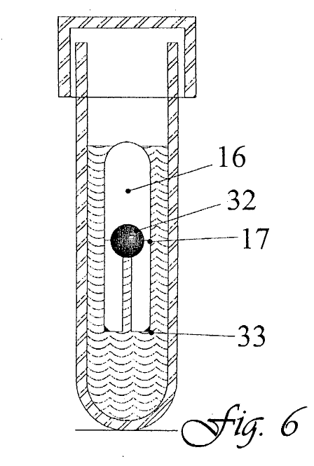

FIG. 4 illustrates the manner in which the relocation of the test device 10 as a result of gas entrapment and elevation 16 can be examined using two lateral light pathways 25 one of which is set in the upper region where the elevating device will intercede and block the light as the said test device rises while the other lateral light pathway is lower down and is blocked by the test device 10 at the start of the testing period but is not blocked when the said device has elevated as a result of gas entrapment. Both light pathways are served by light emitters 26 and the blockage of the light pathways are each determined by separate detectors 27 that record the light intensity being received and allows intelligent systems to determine the time lag to any recognized movement in the test device 10 that would indicate gas entrapment and an increase in the buoyancy for the test device. Under normal operating conditions the most likely characteristics for the light being emitted (from the emitters 26) would be in the red or infra red wave bands. In the event that it is not practical to use these forms of light then FIG. 5 illustrates an alternative sensing system in which the test device 10 is constructed using electrically responsive materials such as iron filings. When the test device elevates 16 then there is a change in the electromagnetic fields 29 being emitted by a single or multiple source of electromagnetic force 28 and detected on the far side of the culture vessel 14 by two sensors (upper 30 and lower 31) that respond to the changes in the field as a result of the shifting shadow where the test device is situated at that time. The time lag to this event as defined by those experienced in the art would be used to quantify the microbial activity.

FIG. 6 illustrates an alternative system for the detection of entrained gas 16 through the admission of a small geometrically suitable low density device 32 into the test device 10. This device 32 would float at the medium: gas interface 17 and would enhance the determination of the volume of the gas entrapped. Such a floating device 32 would allow a more precise determination of the gas production using the light pathway 25 or electromagnetic sensing 29. In order to avoid the risk of the floating device 32 being lost from the test device 10 by the actions of agitation or gravity, the lower rim of said test device would be extended inwards 33 to retain the floatable device 32 within the test device 10.

It will be recognized by those familiar with the art that the present invention may be utilized in a number of manners, each of which will utilize some of the unique features which form a part thereof. Below are some examples of alternative protocols that may be accomplished using the culture chamber.

In the first example of an alternative protocol, the test format is as shown in FIG. 1 except that culture medium 15 only that it is triple concentrated and fills only one third of the defined volume to be eventually be occupied by the liquid medium in combination with the sample in the prepared test format. Another divergence is that the test device 10 would operate within a larger culture chamber 14 that would require the use the extended vanes shown in FIG. 3 as full length vertical vanes 21 extended downwards 24 to keep the test device 10 off the floor of the culture chamber 14. In this example the culture medium 15 is 50 ml of triple strength brilliant green bile growth that is selective for the fermentative activities of the species Escherichia coli, the common indicator organism for health risks in water. This medium is diluted by the addition of 100 ml of the water sample to be tested and the combination of medium and water sample bring the liquid volume contained within the culture chamber 14 in FIG. 1 to an acceptable level above the sunken test device 10 so that the device is fully submerged. At this time when the liquid sample is applied to the said culture chamber, the test device 10 may float up to the surface due to reduced buoyancy resulting from entrained air. This may be corrected by slowly inverting the test device 10 within the sealed culture chamber 14 cap to prevent leakage from the culture chamber during inversion. Such a maneuver of inversion would cause the releases of entrained air from the test device 10 so that the density of said device now exceeds that of the surrounding medium 15. Once the test device 10 has sunk within the liquid medium then the culture chamber 14 is incubated at blood heat for 24 hours. In the event of the fermentative activities of Escherichia coli, the result would follow the form shown in FIG. 2 where the test device 10 has elevated due to entrapment of fermentation gases within the test device 10. Where it is necessary to determine the numbers of Escherichia coli in the water sample then the time lag preceding said elevation can be quantified using either the shift in the interruption of the light pathways 25 illustrated in FIG. 4; or the movement of the shadow imposed by the test device 10 on the electromagnetic forces generated 28 illustrated in FIG. 5. Under normal conditions when testing said water samples the time lag would range from 14,400 seconds for an excessively large population of Escherichia coli related to the presence of billions of cells in the 100 ml water sample down to a single cell if the time lag was 86,400 seconds. By comparative studies against the standard recognized methodologies, a robust and valid methodology can be determined that this relationship exists between the time lag to test device 10 through its elevation in FIG. 2 as a result of the entrapment of fermentation gases 16. This example therefore provides a novel and scientifically defensible method for the determination of Escherichia coli and, through modifying the culture medium 15, other related enteric bacteria that could under, some circumstances pose significant health risks and can generate fermentation gases during incubation, could be detected.

A second example relates to the need to determine the ability of a particular strain of a fermentative bacterium to ferment a range of carbon substrates as a necessary part of the identification of that bacterial strain. To undertake this survey there has to be a multiple of culture chambers of the type shown in FIG. 1 equivalent to the numbers of the said substrates that are to be investigated. Each individual culture chamber 14 contains a test device 10 and the culture medium 15 that is different for each of the substrates to be tested but has the volume illustrated in the FIG. 1. Each medium has a unique but standard formulation for the determination of the range of substrates that would be examined for the generation of detectable fermentation gases. An example of the such a set of fermentation results typical for a strain of Escherichia coli is (bracket shows G for gas detected by test device 10 elevating as a result of fermentation gas accumulation 16); and ND indicates that gas fermentation was not detected): Glucose, G; Lactose, G; Sorbitol, G; Adonitol, ND; and Inositol, ND. After the incubation period it is possible to view the position of the test devices 10 in each of the culture chambers 14 and determine the fermentative ability of the bacterial strain being tested as an aid to the identification of that strain of bacterium.

As can be seen from the foregoing description, the present invention involves a number of stages in the conductance of the test procedure which generates advantages both from the perspective of user convenience and also from the perspective of improving the resolution of the impact of the selective culture medium 15 on the targeted organism. For the user, the procedure using the devices described above, these inventions can allow the examination of the given sample through incubation to generate clear and distinct visual evidence of the activities of the organism being determined by the physical relocation of the test device 10 and the time lag to that event after the start of the incubation of the inoculated culture chamber allows the quantification of the numbers of the targeted organism in the sample under examination. Where the user desires to determine the range of substrates that may be fermented with the production of entrapped gases recorded by the elevation of the test device 10 with a culture chamber 14 where at least one said cultural apparatus is devoted to each of the substrates for examination using a suitable culture medium 15 then there is the potential to use the fermentation of gases to aid in the determination of the organism.

By way of a quantitative example of the use of the culture chamber 14 containing the test device 10 in a manner appropriate to the descriptions of this invention, the application of the technique will be described for the determination of the presence or absence of total enteric bacteria in a given water sample. For this example the culture vessel would have a sufficient capacity to hold 100 ml of the water sample for examination and 50 ml of triple strength Lauryl Tryptose broth to determine the fermentative activities of any total coliform bacteria present within the said sample. To assure evidence of entrapped fermentation gases within the test device 10, the sealed culture chamber 14 needs to be inverted after the sample has been added to the medium to evacuate any headspace air from the device so that the said device will sink. For effective determinations of any fermentative activities generating gases, the test device 10 should have a height less than two thirds of the height of the liquids added to the culture vessel 14. Incubation would be at blood heat (37±1° C.) and a positive detection of the total coliform bacteria would be the elevation of the test device 10 within the liquid medium 15 to create an image similar to that shown in FIG. 2 the time of which can now be reported by direct visual observation or by the use of spectrophotometric techniques (FIG. 4) or electromagnetic inferences (FIG. 5). By a calculation of the time lag as being that difference between the start of the incubation period of the investigation and the time to the first observation of the elevation of the test device 10. An inverse relationship exists between the length of the time lag commonly measured in seconds and the population of total coliform bacteria measured in total coliform bacterial cells per 100 ml. One such equation, by nature of an example that would link the time lag (TL) to the total enteric bacterial population (TEP), is:

TEP=A*B

Where A is (Power 10(75,600)−TL) and B is (0.0001389+(0.00000002273*(TL−32,400). It will of course be understood by those familiar with the art that the particular embodiment of the invention presented are by way of illustration only, and are meant to be in no way restrictive; therefore, numerous changes and modifications may be made, and the full use of equivalents reported to, without departing from the spirit of the scope of the invention as outlined in the appended claims.

I claim:

1. A test apparatus, for use in the determination of fermentative activities of a targeted microorganism within a given sample, in which said test apparatus comprises a vertical test device sealed on the top side and open at the bottom with open slots up the sides to allow a free exchange of microorganisms and chemicals between the said the test device and the culture chamber within which the test device is sunken by the nature of the heavier designed density of the test device in comparison to the culturing fluids within the culture chamber and in addition that the test device would cause the collection of some of the gases resulting from any subsequent fermentative activity in a manner that would lead to a fall in the overall density of the test device causing said device to develop a density equal to, and then less than, the density of the fluids in the culture chamber thus causing the device to rise up to the surface whereupon the relocation of the said device would be interpreted as indicating the production of fermentation gases within the culture chamber.

2. A test apparatus as claimed in claim 1, and further including a means to detect optically or electromagnetically the moment at which the device floats up in the culturing fluid to the surface as a direct result of fermentation gases being entrained within the test device causing a sufficient loss in density to the device along with the corresponding gain in buoyancy in a manner that the time lag between the start of the incubation of the test using the test device within a culture chamber can be used to determine the size of the active fermenting microorganisms present within the culturing fluid as generating an inverse relationship between the time lag and the population of targeted microorganisms in the sample being examined.

3. A test apparatus, as claimed in claim 1, and further including the addition of lateral vanes that extend out for a part, or the whole, of the vertical length of the device in a manner that can be mountable in such a manner that the vanes would possess a density that when complemented with the density of the central form of the test device would assure that the test device would have a sufficient density to sink in a given culturing fluid through possessing a predictable density that would be the product of the culture medium and the sample added to the culture chamber for examination, and, in addition, the attached vanes would aid to render the apparatus vertical in a manner that would expedite travel within the culture chamber as the buoyancy of the device is compromised by the fermentation gases where the culture chamber had a sufficiently larger diameter than the test device that the culture chamber would fail to keep the test device vertical without the presence of the vanes.

4. A test apparatus, as claimed in claim 1, and further including an opaque floatable device within the apparatus that would mark the volume of fermentation gases collected in the apparatus that would be seen as a shadow when light or an electromagnetic field was generated through the culture chamber and a shadow created by the test device was detected to indicate the location of the test device within the culture chamber.

5. A method for testing a sample for the presence of targeted microorganisms capable of causing the fermentation upon incubation involving the use of specific and diagnostic chemicals considered to have the potential for the production of gaseous end products to be achieved by placing a test device within a culture chamber, said test device having been inverted so that the closed end is uppermost and the open end is lowermost to assure that the device through having a density greater than the fluid culture allows through various openings in the base and in the lower parts of the vertical walls such microorganisms as may be within the culturing fluid to move throughout the fluids and to potentially ferment these specific chemicals to gaseous end products thereby causing movement in the test device involving a gas-induced relocation that can then be used for diagnostic purposes in quantitative and qualitative manners presently commonly achieved using the Durham's tube.

6. A method as claimed in claim 5 and further including a multiplicity of culture media each of which possesses different potentially fermentable organic compounds may use the said elevation of the test device to indicate which of the said compounds are fermentable through the production of entrained gases so that such information may then be interpreted using known methods.

7. A method as claimed in claim 6 but having a single dilution of the sample within a fermentable culture medium and, upon incubation, the time lag to the elevation of the test device is used to give quantifiable information that can, upon statistical interpretation, generate an estimate of the population size as a possible population number in the sample examined.

8. A method as claimed in claim 1 further may have a said configuration in which the test device will occupy and entrap a void fluid volume within the culture chamber and the void volume will form a part of the total fluid volume within the culture chamber and that the ratio of the void fluid volume to the total fluid volume will influence the time lag to the elevation of test device, detected by any means, in a manner wherein the smaller the fraction of total fluid volume that is in the void volume within the test device then the longer would be the time lag to the declaration of a positive detection of fermentable gases through said elevation of the device and so said fraction would be standardized in the operation of a particular procedure.

9. A method as claimed in 8, and further including the use of vanes to the outside of the test device in a manner that would allow the test device to remain vertical where there is a significantly larger total fluid volume in the culture chamber than void fluid volume in the test device.

10. A method as claimed in 9, and further including that the vanes may be removable and constructed of a different density of material such that the final density of the test device to which the vanes are attached becomes adjusted to a degree significantly greater than that of the defined inoculated culture medium including any liquid samples added so the, when voided of any entrapped air or gases, the test device will sink within the culturable fluids of the culture chamber.
